(19) United States
(12) Patent Application Publication
(10) Pub. No.: US 2002/0121450 A1
(43) Pub. Date: Sep. 5, 2002

(54) Genetic preservation kit

(76) Inventors: Juan Ricardo Inga, Allentown, PA (US); Linda Carol Baker, Pittsburgh, PA (US)

Correspondence Address: Thomas C. Wettach, Cohen & Grigsby, P.C., 11 Stanwix Street, 15th Floor, Pittsburgh, PA, 15222, US

(21) Appl. No.: 09/798,102

(22) Filed: Mar. 2, 2001

(51) Int. Cl. A61L 15/00; A61B 19/02

(52) U.S. Cl. 206/438

(57) ABSTRACT

A genetic survival kit made up of a plurality of sealable vials which are supported in a secure support. The vials each contain an oxygen adsorbent and a desiccant. A cushioning material may also be included in the vials to support the placement of a genetic sample.

### FIELD OF THE INVENTION

The present invention relates to a kit for preserving genetic information of individuals or animals, and in particular to a kit for preserving genetic samples associated with individuals which are collected in home.

### BACKGROUND OF THE INVENTION

It is becoming more common for individuals to collect and preserve genetic samples of infants and family members for use in the event a family member is abducted or otherwise becomes missing. This information, if available, can be the critical link to evidence that could help identify, locate and possibly save the life of a missing person. Hair sample comparison and DNA print matching, for example, can be used by forensic pathologists to identify an unknown hair, blood, or tissue sample as that of a missing person, thereby generating a lead to locating such person. More recently, the collection and storage of genetic materials has taken on significant importance with the linking of disease to genetic abnormalities or precursors. Many disease and disorders are the result of disorder of cellular functions. In addition, storing the genetic materials at infancy provide: 1) a baseline before disease or disorders occur, 2) effects of radiation, and 3) mutations due to chemicals. However, genetic materials such as stem cells are more easily attainable with infants than adults.

A number of methods for the collection of genetic material have been proposed, see, e.g., U.S. Pat. Nos. 5,101,970 and 5,856,102. The prior art teaches methods for the collection and storage of DNA and other genetic materials. Most of the recent methods for home use or self storage involves placing the samples in containers which are located in refrigerated units or freezers. This is similar to commercial methods that have been used for many years in which the genetic material is cryogenically frozen. In some home methods, storage of the genetic material is in the form of a small container, such as an envelope that can be placed in a safe place, such as safety deposit box.

While many of these methods purport to provide genetically stable specimens over a long period of time, many do not keep the specimens safe from the effects of the environment, especially humidity and oxidation reactions. Accordingly, it is an object of the present invention to provide a self contained kit for the safe storage of collected genetic material. It is a further object of the invention to provide a contaminant free or substantially free self container which provides long-life storage of genetic materials. It is yet another object of the invention to provide a genetic preservation kit for storage of self collected genetic materials. It is still another object of the invention to provide a genetic material preservation kit for storing multiple genetic samples without refrigeration or cryogenic procedures. Other advantages will be apparent from the description of the present invention.

### SUMMARY OF THE INVENTION

The present invention comprises a system for the storage of samples of the genetic materials. In a preferred embodiment of the invention, a storage system is provided that contains a support medium, preferably of a resilient material having a plurality of receptacle slots to securely hold receptacles containing collected samples of genetic material. In the preferred embodiment, the samples to be collected and stored include blood, placenta or umbilical cord (stem cells), hair (preferably including the root), nail clippings, mucous, a tooth or teeth (typically baby teeth), and semen. It is preferred that samplings are from an infant at or shortly after birth. However, adults can take advantage of this system, but must recognize that stem cell recovery is usually obtained from the umbilical cord at birth.

In addition to the storage support medium, the storage system includes a plurality of storage receptacles such as glass or plastic vials in which each specimen is stored. Each glass or plastic vial includes a sealable lid or cover, a desiccant, an oxygen adsorber and an absorbent material. The specimens are placed in their respect vials and sealed therein by placing a sealable lid over the opening in the vial. The sealed vials are then placed in an associated receptacle slot in the storage support medium. In a preferred embodiment, the storage support medium is placed in a metal foil or plastic-metal foil wrapping material. The package is then stored for safe keeping in any convenient storage location.

The unique storage system of the present invention requires no refrigeration and prevents oxygen and moisture contamination of the samples. Most importantly, the storage system of the present invention provides a viable alternative to expensive commercial cryogenic storage of genetic materials for long term future use. Also, the present invention is readily adaptable to home use and collection techniques. Other advantages of the present invention will become apparent from a perusal of the following detailed description of a presently preferred embodiment taken in connection with the accompanying drawings.

### BRIEF DESCRIPTION OF THE DRAWING

FIG. 1 is a perspective view of the support medium of the present invention for containing a plurality of specimen vials; and

FIG. 2 is a perspective view of a presently preferred vial and contents.

### PRESENTLY PREFERRED EMBODIMENT

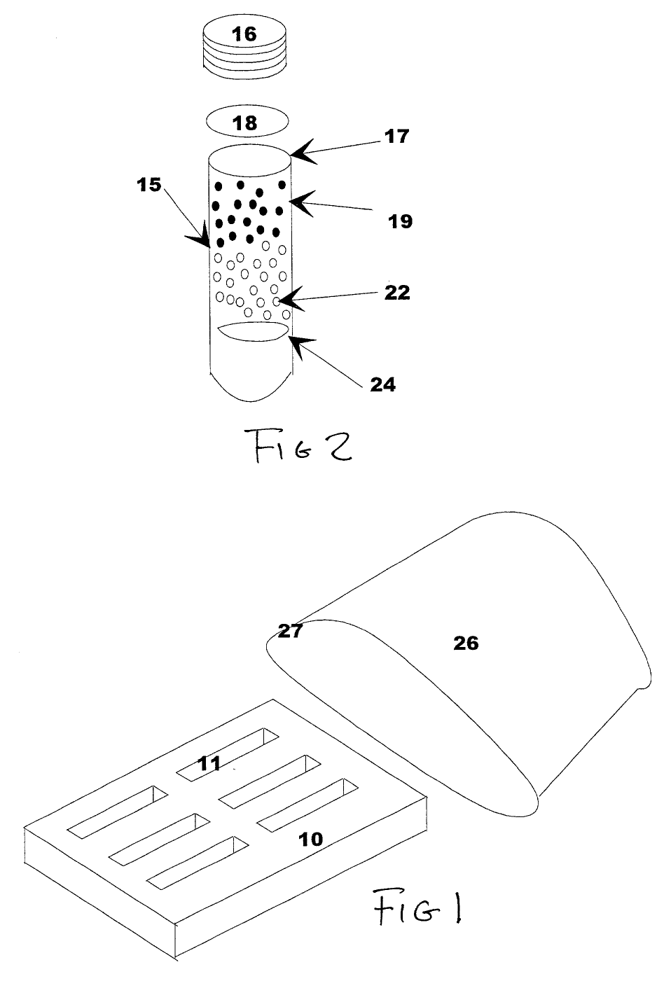

Referring to FIG. 1, a support medium 10 is shown having a plurality of vial receptacles 11. Each vial receptacle is dimensioned to engagingly receive an associated receptacle. Preferably, support medium 10 is made from a foamed plastic such as polystyrene or a molded rubber material to provide cushioning or impact resistant.

Vial 15, as shown in FIG. 2, includes a lid or cap 16 which sealingly engages the periphery of vial opening 17. Additionally, a seal 18 may be placed within opening 17 to provide a hermetic seal. Vial 15 is preferably made of glass or plastic.

An oxygen absorber 19, such as fine divided iron oxide, is placed within the vial in quantities sufficient to absorb free oxygen from the air around them and chemically bind it by oxidizing the finely divided iron into iron oxide. By removing oxygen from the inside of vial 15 when the specimen is placed therein, it prevents oxidative rancidity, free radical breakdown and respiration by insects, fungi or aerobic bacteria. Thus, the removal of the free oxygen from vial 15 extends the storage life of the specimens. By sealing the contents with sealing cap 16, oxygen ingress is further retarded. In a preferred kit, 50 cc vials are used for storage. In such vials, a 2 to 5 gram sample is placed along with 10 cc of a selected oxygen absorber and 20 grams of desiccant 22.

Desiccant 22 is either a natural or synthetic desiccant used for controlling humidity/moisture within vial 15. In a preferred embodiment, the following desiccants are suitable for use:

Montmorillonite clay (a naturally desiccant composed primarily of magnesium aluminum silicate and preferred for low room temperature storage, but is not preferred for storage at temperatures above about 120° F.); Lithium Chloride; Activated alumina: (Activated alumina is a preferred desiccant for the present invention); Molecular sieves such as:

alkali alumino-silicate;

DQ11 Briquettes;

Silica Gel Beads; and

Calcium Oxide.

Commercial desiccant can also be used as well as dried wood.

Cushioning material 24 may also be included to protect the specimen. Preferably, cushioning 24 is a foam material and is positioned in the bottom of vials 15.

A cover or sleeve container 26 is used to cover and protect support 10 and vials 15 contained in receptacles 11. Sleeve 26 is preferably made from metal or plastic having an opening 27 into which support 10 can be placed. The periphery of opening 27 preferably has an adhesive to maintain the support in sealed closed storage condition. Sleeve 26 further protects the samples from ultraviolet radiation and harmful gases.

As can be seen the present invention provides a low cost alternative to otherwise expensive cryogenic storage techniques. The presently preferred embodiment of the invention will provide safe, long life storage of genetic samples. While presently preferred embodiment has been shown and described in particularity, the invention may be otherwise embodied within the scope of the appended claims.

What is claimed:

1. A genetic survival kit comprising a plurality of sealable vials, a support medium having a plurality of receptacles at least equal in number to the number of sealable vials, said receptacles being dimension to engageably contain a sealable vial, each of said vials having a removable resealable cap and containing an oxygen absorbent and a desiccant.

2. A genetic survival kit as set forth in claim 1 including a closeable sleeve dimensioned to contain said support medium having plurality of sealable vials engaged within its receptacles.

3. A genetic survival kit as set forth in claim 1 or 2 including a cushioning material in at least one of said vials.

4. A genetic survival kit as set forth in claim 1 or 2 wherein said oxygen absorbent is finely divided iron.

5. A genetic survival kit as set forth in claim 1 or 2 wherein said desiccant is activated alumina or silica gel.

6. A genetic survival kit as set forth in claim 1 or 2 wherein said support medium comprises a resilient material.

7. A genetic survival kit as set forth in claim 1 or 2 wherein said sealable vials comprise glass or plastic.
